(19) United States
(12) Patent Application Publication
(10) Pub. No.: US 2012/0210516 A1
(43) Pub. Date: Aug. 23, 2012

(54) TRAVEL PILLOW

(76) Inventor: Stevan Popovic, Troy, MI (US)

(21) Appl. No.: 13/398,977

(22) Filed: Feb. 17, 2012

Related U.S. Application Data
(60) Provisional application No. 61/443,860, filed on Feb. 17, 2011.

(51) Int. Cl. A47G 9/10; A61F 9/04

(52) U.S. Cl. 5/640; 5/636

(57) ABSTRACT

A travel pillow comprises a pliable eye mask which is contoured for securement over a user's eyes. Means for securing are provided to secure the eye mask over the user's eyes. The travel pillow also includes a pair of ear pillows. Each of the ear pillows is adjustably secured to the means for securing and positioned on a respective side of the eye mask so that the ear pillows conceal the user's ears when the eye mask is worn by the user. The travel pillow also includes at least one neck pillow that is adjustably secured to the means for securing. The neck pillow includes an upper section which is thin and pliable, and a lower section which is filled with a stuffing material to provide support to the user's neck. The lower section can be positioned as desired by the user to provide adequate comfort and support.

### CROSS REFERENCE TO RELATED APPLICATION

This application claims the benefit of U.S. Provisional Application 61/443,860, which was filed on Feb. 17, 2011, the disclosure of which is hereby incorporated by reference.

### BACKGROUND OF THE INVENTION

1. Field of the Invention

The present invention pertains to a travel pillow. More particularly, the present invention pertains to a combination travel pillow and eye mask.

2. Description of the Prior Art

Travel pillows are well-known in the art, such as disclosed by U.S. Pat. No. 4,097,086 to Hudson. The pillow disclosed by Hudson includes a hood for fitting over the top of a chair backrest. The hood includes straps which attach to a pillow holder containing a pillow, and the pillow holder has straps for extending over the user's shoulder for support. The device disclosed by Hudson allows the user to rest their head against the hood and to secure the pillow in position with respect to the hood such that the user can rest their head against the pillow while they sleep.

In addition, U.S. Pat. No. 4,031,578 to Sweeney et al. discloses a contoured travel pillow which cushions the user's back and head in a cavity and provides support against “head-rolling” which usually results when a traveler attempts to sleep while sitting in a generally upright position.

The patents to Hudson and Sweeney attempt to provide adequate neck and head support to the user while the user sleeps in a generally vertical orientation, such as in a plane, car, or train. Notwithstanding their ability to accomplish this goal, these patents are unable to block light or sound while the user is sleeping.

U.S. Patent Application No. 2008/0216244 to Minton discloses a combination pillow and eye mask that also includes personal speakers. The device includes three pillows connected end-to-end which support the back and each side of the user's head. The pillows positioned on each side include a recess having a speaker positioned therein. Each speaker can be configured to produce any variety of sound, including white noise, ocean sounds, rain, music, or the like. The three pillows are encircled by a strap to secure the pillows around the user's head. The front portion of the strap extending across the user's face includes an eye mask for blocking light. Although this device provides a pillow, an eye mask, and speakers, it is intended for use in a horizontal position and would not be desirable for travel purposes where the user is sleeping in a generally upright position. This is because the pillows are not adjustable and provide support only to the user's head, whereas a suitable travel pillow preferably provides most of its support to the user's neck.

Thus, there remains a need for a travel pillow which includes an eye mask and which provides ample neck support so that it can be used in a generally vertical orientation.

The present invention, as is detailed hereinbelow, seeks to fill this need by providing a travel pillow including an eye mask, adjustable ear pillows, and a neck pillow secured thereto which is positionally adjustable with respect to the eye mask so that the user can comfortably position the neck pillow with respect to his or her head and neck.

### SUMMARY OF THE INVENTION

The present invention provides a travel pillow which generally comprises:
(a) a pliable eye mask contoured for securement over a user's eyes;
(b) means for securing the eye mask over the user's eyes;
(c) a pair of ear pillows, each ear pillow being adjustably secured to the means for securing and positioned on a respective side of the eye mask such that the ear pillows conceal the user's ears when the eye mask is worn by the user; and
(d) at least one neck pillow adjustably secured to the means for securing.

For a more complete understanding of the present invention, reference is made to the following detailed description and accompanying drawings. In the drawings, like reference characters refer to like parts throughout the views in which:

### BRIEF DESCRIPTION OF THE DRAWINGS

FIG. 1 is a perspective view of a first embodiment of the present invention hereof;

FIG. 2 is a perspective view showing the eye mask, the ear pillows, and the top of the neck pillow for securement around a user's head;

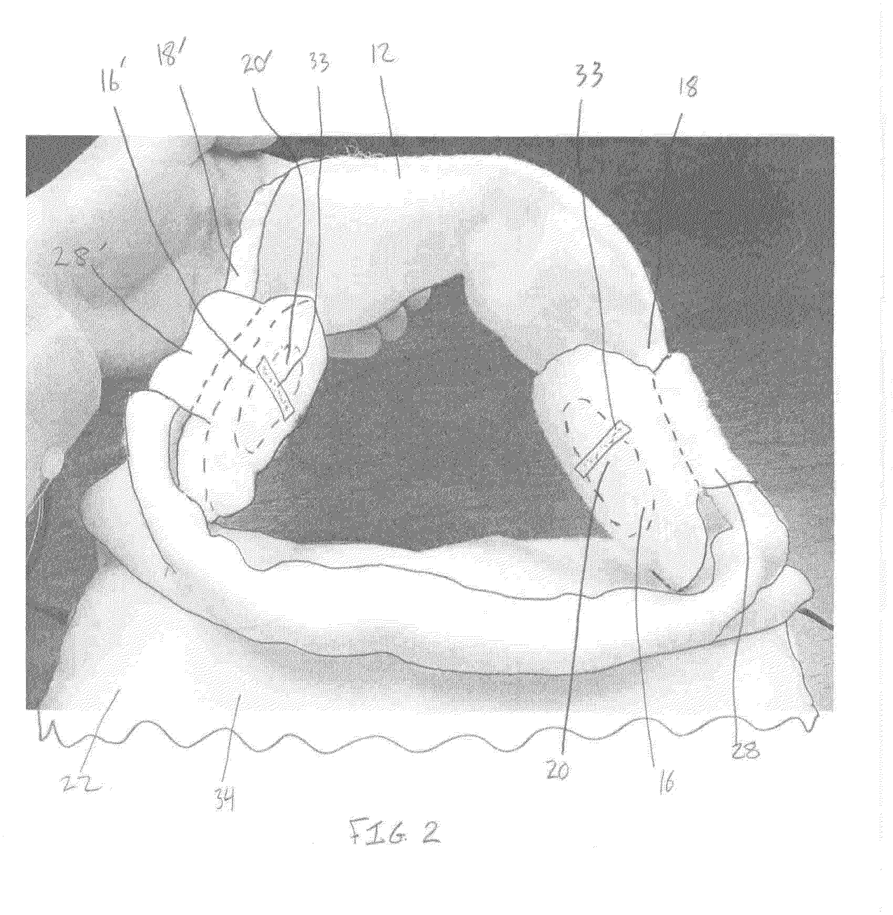

FIG. 3 is an enlarged view showing the means for securing the eye mask, the ear pillow, and the top portion of the neck pillow together;

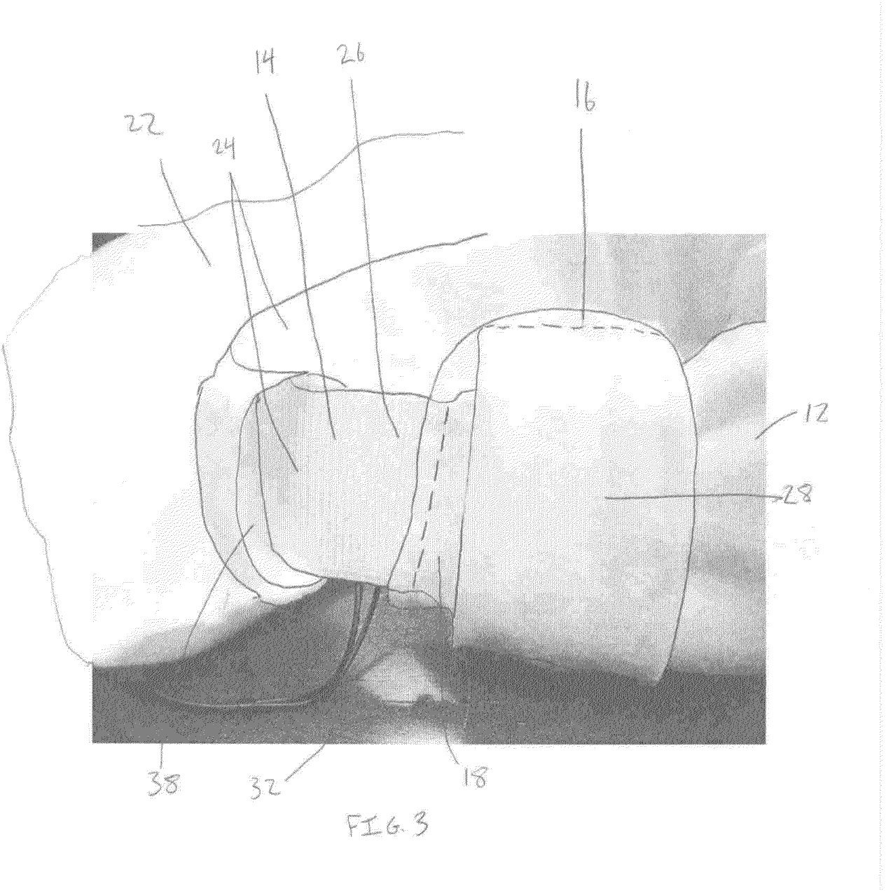

FIG. 4 is an enlarged view showing the back of the ear pillow having a speaker secured therein;

FIG. 5 is a perspective view of the back portion of the present invention showing the travel pillow in an elongated position;

FIG. 6 is a perspective view of the travel pillow showing the lower section of the neck pillow folded under the upper section of the neck pillow; and

FIG. 7 is a perspective view showing the means for retaining the earbuds, or earphones, to the ear pillows.

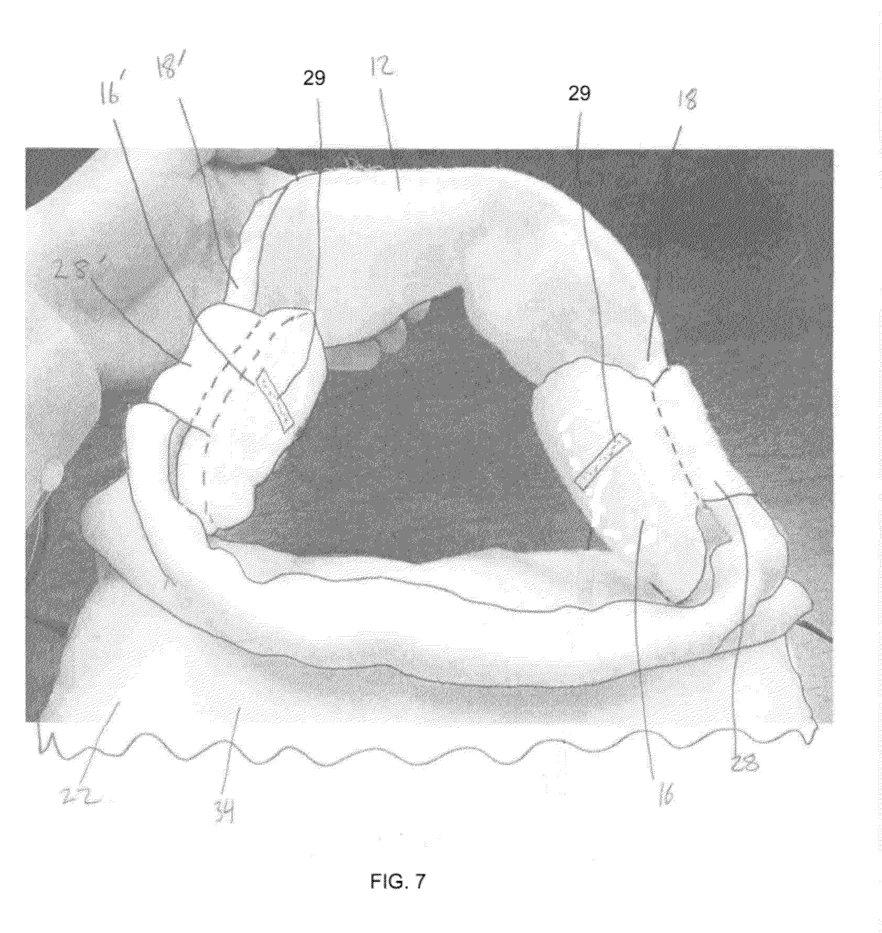

### DETAILED DESCRIPTION OF THE PREFERRED EMBODIMENT

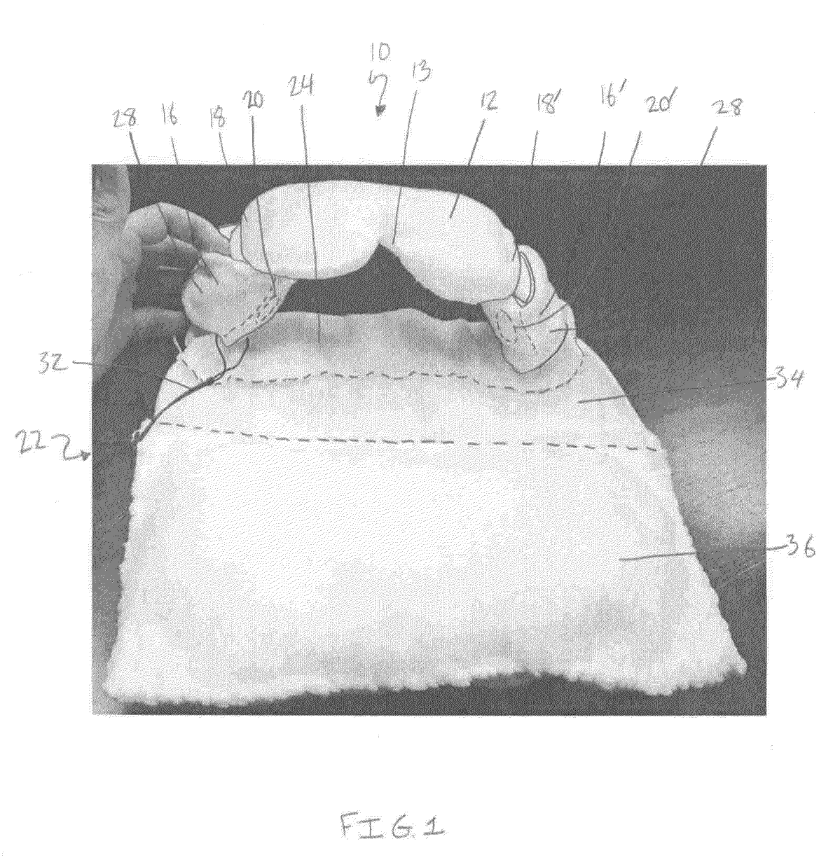

In accordance with the present invention and as shown generally in FIG. 1, there is provided a travel pillow 10 which generally comprises: (a) a pliable eye mask 12 contoured for securement over a user's eyes, the eye mask 12 including a flexible band 14 for surrounding the user's head and securing the eye mask 12 over the user's eyes; (b) a pair of ear pillows 16,16′, each ear pillow 16 being adjustably secured to the flexible band 14 and positioned on a respective side 18 of the eye mask 12 such that the ear pillows 16,16′ conceal the user's ears when the eye mask 12 is worn by the user; and (c) at least one neck pillow 22 adjustably secured to the flexible band 14.

At the onset, it should be noted that much, if not all, of the components which comprise the present invention are preferably formed from a soft pliable material which is comfortable, soothing to the touch, and sleep-inducing. Any suitable materials which are well-known to one having ordinary skill in the art can be used, including fabrics (made from both natural and artificial fibers) such as cotton, satin, flannel, fleece, polyester blends, or the like. The pillow 10 can be formed from a number of different materials or a single uniform material. A preferable material type is the Spandex-type material which is currently used with many personal products, such as pillows. These materials are often Nylon/Spandex or polyester/Spandex blends (typically about 85% Nylon/polyester and about 15% Spandex). This material is desirable for use because it is stretchable, breathable, and durable.

Furthermore, many of the components, and in particular, the eye mask 12, ear pillows 16,16′, and neck pillow 22, are soft and supple and include stuffing or other suitable filler (e.g., memory foam, micro polystyrene beads, or the like) to provide additional comfort to the user.

In addition, the fabrics and the various components discussed below are secured together using suitable means which are well-known in the art. Preferably, the fabrics are sewn together.

The eye mask 12 is contoured in a manner similar to typical eye masks which are well-known in the art, and includes a cutout portion 13 to accommodate the user's nose. The eye mask 12 is sufficiently large to block out the ambient light. As discussed above, the eye mask 12 is formed from any suitable soft and comfortable material and preferably includes stuffing or other suitable filler material.

Means for securing 24 the eye mask 12 to the user's face is included as well. Preferably the means for securing 24 comprises an elastic or flexible resilient band 14 having opposed ends 26,26′, each of the ends 26,26′ being secured to a respective side 18,18′ of the eye mask 12. The means for securing 24 can alternatively comprise an elastomeric strip which may be enclosed in fabric or any other suitable flexible band as understood by one having ordinary skill in the art. The means for securing 24 can further include any suitable type of releasable clasp (not shown) to allow the user to strap the eye mask 12 over their eyes rather than pull the eye mask 12 down over their head. When a releasable clasp is provided, the means for securing 24 does not necessarily need to be flexible (although it still can be). In this instance, the means for securing 24 can then also include any type of strap or connector which is suitable for use herewith, such as one which is not elastic in nature. The means for securing 24 can also include a clasp, buckle, or other suitable mechanism for adjusting the length of the inelastic band. The means for securing 24 the eye mask 12 are also connected to the neck pillow 22 in any manner described in further detail below.

The travel pillow 10 additionally includes the pair of ear pillows 16,16′. Each ear pillow 16 is adjustably secured to the eye mask 12 and/or the means for securing 24 to allow the user to comfortably adjust the ear pillows 16,16′ to his or her face. As discussed above, each ear pillow 16 is formed from a soft and comfortable material and preferably includes stuffing or other filler material to provide additional comfort to the user. Each of the ear pillows 16,16′ comprises any suitable shape for use herewith, including a generally circular shape, a shape contoured to an ear, or the like. Preferably, each of the ear pillows 16,16′ includes a respective retaining strip 28,28′ which is secured to the back of the ear pillow 16 at its top and bottom. The retaining strip 28 forms an enclosed area for retaining the means for securing 24 therein. In this regard, each ear pillow 16 can be adjusted along the length of the means for securing 24 to accommodate the size and shape of the user's head.

Preferably, the travel pillow 10 is used in conjunction with earphones, or earbuds which are connected to an audio source, or input, such as an MP3 player like an IPod®, or any other suitable type of audio input device. Each ear pillow 16,16′ can include means for retaining 29 the earbud to the ear pillow 16. The means for retaining 29 are provided on each ear pillow 16,16′ and can be positioned on the side of the ear pillow which abuts the user's face, the rearward side thereof, or anywhere else which is desirable. The means for retaining 29 can comprise a strip of fabric which is secured to the ear pillow 16 at its ends and having the earbud and wire passing therethrough. The means for retaining 29 could otherwise include any suitable type of clip or retaining mechanism for engaging the earbud or wire. The means for retaining 29 is provided to retain the earphone (earbud) somewhat in position if it falls out of the user's ear during use. This has particular utility when the user falls asleep and the earphone falls out. It is apparent that retaining the earphone in a location near the ear pillows 16,16′ is desirable to keep the earphones from getting lost, dirty, or damaged.

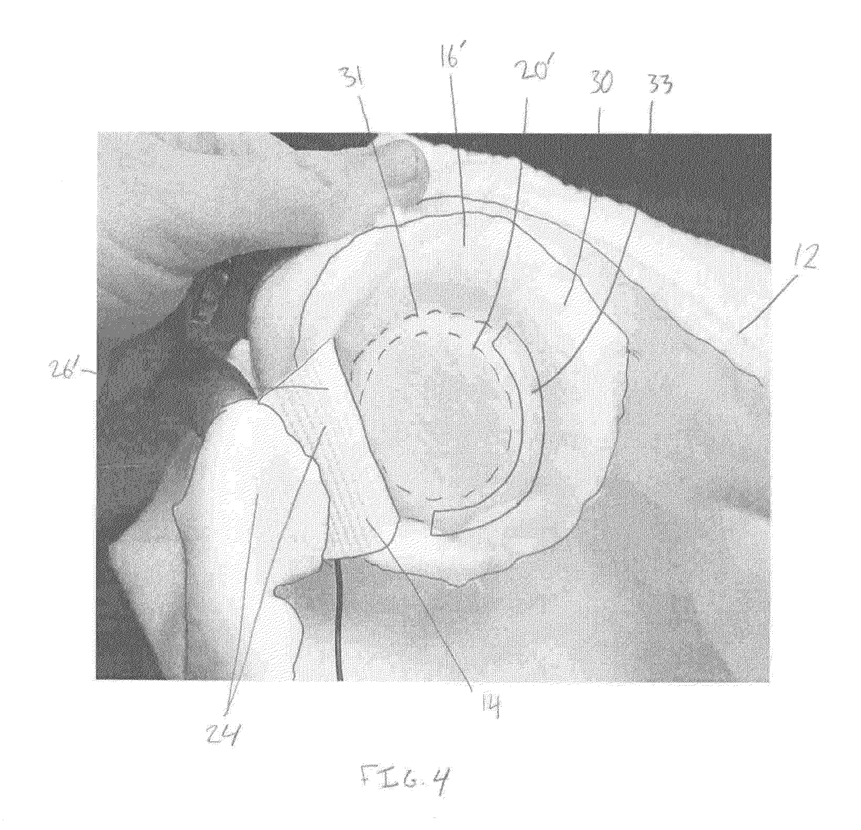

Alternatively, each ear pillow 16,16′ can include a pocket or any other suitable means for retaining a speaker 20. As shown in FIG. 4, when the speaker 20 is provided, it can be sewn into the back 30 of the ear pillow 16 and/or embedded within the stuffing. Alternatively, the ear pillow 16 can include a closable pocket 31 for retaining the speaker 20. The pocket 31 can include any suitable closure means 33 including a zipper, a button, a hook-and-loop fastener (e.g., Velcro®), or the like. Preferably, the speaker 20 can be removed from the ear pillow 16 so that the travel pillow 10 can then be washed.

The speaker 20 can comprise any suitable personal speaker well-known in the art which is sufficiently small, quiet, and energy efficient (such as for use with a battery-powered device). Any speaker used with commercially-available headphones is suitable for use herewith.

Each speaker 20 further includes means for receiving 32 a signal used to generate sound. The means for receiving 32 a signal can comprise speaker wire, a wireless connection, or the like. Preferably the means for receiving 32 comprises a wire having a suitable connector for use with any suitable input device, such as an iPod®. The means for receiving 32 can also include any suitable wireless connection which is commercially available and utilizes known technology, such as radio waves (e.g., a short-range radio signal generator, a signal from a wireless LAN, Bluetooth®, etc.), and so forth. It is contemplated that any suitable new wireless technology which becomes commercially available can be suitable for use herewith.

The ear pillows 16,16′ are soft, pliable, and comfortable for use even during long periods of time. Even more so, when the speaker 20 is disposed within the ear pillow 16, the ear pillow 16 and speaker 20 combination provides a speaker which is adjustably positioned adjacent to the user's ears—opposed to an earbud which is positioned in the ear canal, or a set of headphones secured over the user's head and ears.

The travel pillow 10 additionally includes at least one neck pillow 22 adjustably secured to the eye mask 12. As discussed above, the neck pillow 22 is formed from a soft and comfortable material and preferably includes a portion having stuffing or other suitable filler material. Preferably, the neck pillow 22 is generally rectangular in shape and includes a thin upper section 34 and a plush, or stuffed, lower section 36.

The upper section 34 is preferably secured to the means for securing 24. More preferably, the upper section 34 is secured to the elastic band 14 (when provided). Even more preferably, the upper section 34 includes an elongated loop 38 section which retains the means for securing 24 extending therethrough. The upper section 34 can be permanently secured to the means for securing 24, such as having the loop 38 being sewn together. Alternatively, the upper section 34 can be removable from the means for securing 24, such as the loop 38 having an edge which is held together by a removable fastener like a hook-and-loop (Velcro®). In this regard, the neck pillow 22 can be removed from the eye mask 12 and ear pillows 16,16′ when desired by the user. For instance, when the user is laying horizontally in bed and there is no need for the neck pillow 22.

The thin upper section 34 is pliable, positionable, or can be folded or otherwise manipulated to allow the stuffed lower section 36 to be adjustably positioned with respect to the user's neck for maximum comfort level. The lower section 36 can form any suitable shape, such as cylindrical, rectangular, horseshoe-shaped (to assist also with lateral support), or the like.

Optionally, the neck pillow 22 can be removable from the means for securing 24 so that the neck pillow 22 is detachable from the travel pillow 10. This may be desirable for some particular uses of the travel pillow 10 when the neck pillow 22 is not needed, such as when a user uses the eye mask 12 and ear pillows 16,16′ in bed. In this instance, the neck pillow 22 and the means for securing 24 can include any suitable means for releasable attachment to each other (not shown), such as a zipper, buttons, hook-and-loop fasteners, or the like.

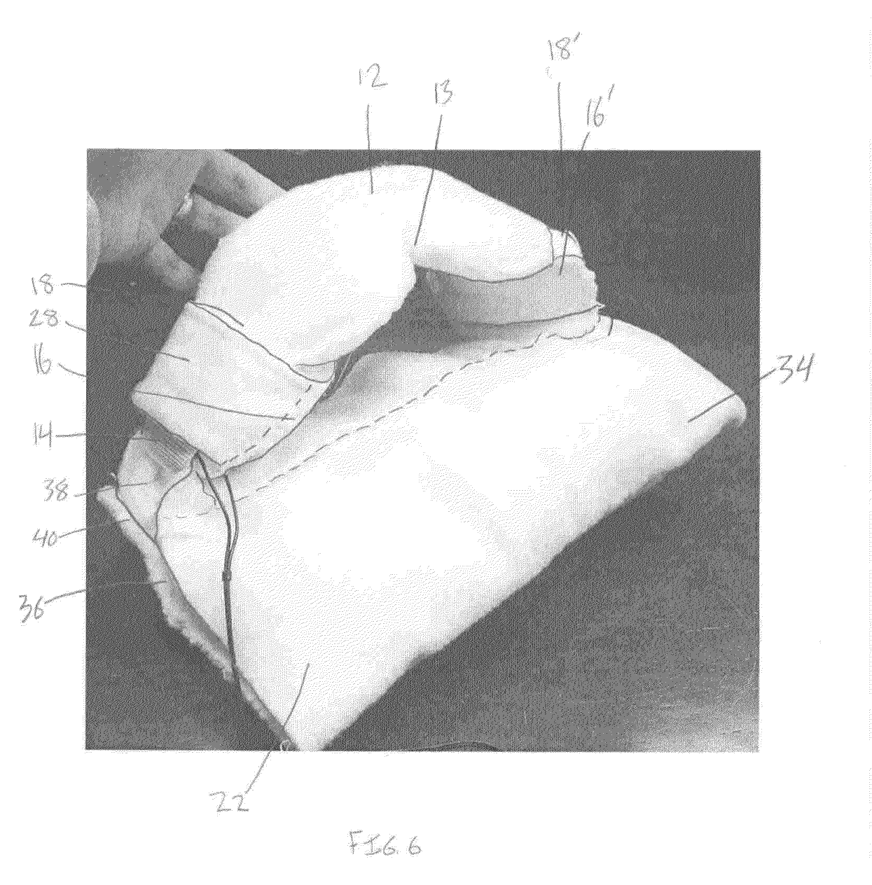

Optionally, the travel pillow 10 can include means for retaining 40 the neck pillow 22 in position, such as during use or storage. The means for retaining 40 can comprise any suitable fastener, such as buttons, hook-and-loop fasteners, and so forth. The means for retaining 40 can be positioned on the neck pillow 22 and/or the elastic band 14 to retain the travel pillow 10 in any desirable position, such as shown in FIG. 6.

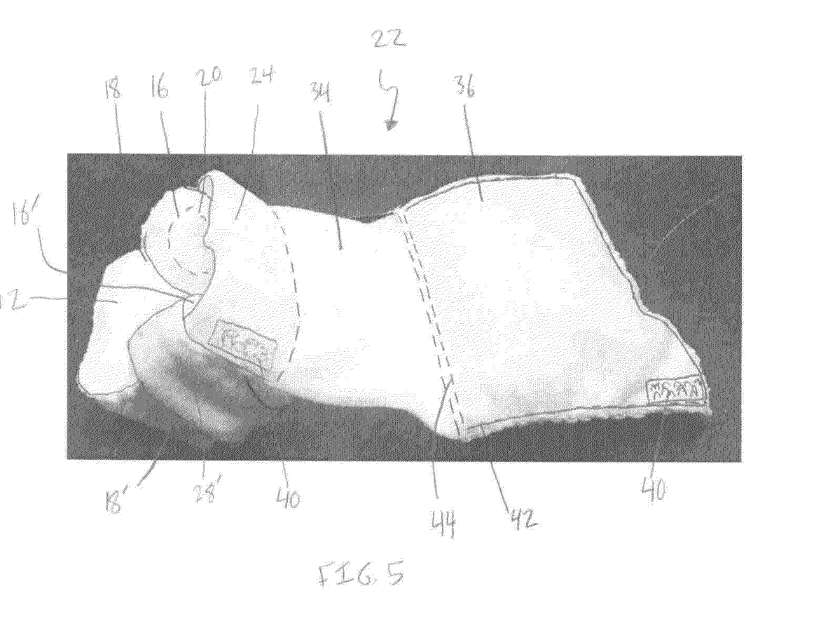

As shown in FIG. 5, the neck pillow 22 can further optionally include a pocket 42 for stowing the eye mask 12 and the ear pillow 16,16′ therein when not in use. The pocket 42 is preferably disposed on a side (either front or back) of the lower section 36 of the neck pillow 22. Preferably the pocket 42 has three sealed edges and one edge 44 which is either unsealed or closable via a suitable fastener such as a zipper, buttons, hook-and-loop fasteners, or the like. When the travel pillow 10 is not in use, the eye mask 12, ear pillows 16,16′, the means for securing 24, and the upper section 34 can be rolled up and stored in the pocket 42. It should be noted that the input device (e.g., iPod® or other suitable MP3 player) can be stowed in the pocket 42 while is use, thereby ensuring that the input device is not loose while the user is asleep or resting.

According to the invention described above, a travel pillow is provided which is comfortable, eliminates ambient light and sound, and which provides ample support to the user's head and neck allowing the user to relax and/or sleep in a generally vertical orientation.

As is apparent from the preceding, the present invention provides a travel pillow including an eye mask, ear pillows, and a neck pillow secured thereto which is positionally adjustable with respect to the eye mask so that the user can comfortably position the neck pillow with respect to his or her head and neck.

What is claimed is:

1. A travel pillow comprising:
(a) a pliable eye mask contoured for securement over a user's eyes;
(b) means for securing the eye mask over the user's eyes;
(c) a pair of ear pillows, each ear pillow being adjustably secured to the means for securing and positioned on a respective side of the eye mask such that the ear pillows conceal the user's ears when the eye mask is worn by the user; and
(d) at least one neck pillow adjustably secured to the means for securing.

2. The travel pillow of claim 1 wherein the means for securing comprises a flexible band having opposed ends, each end of the band being connected to a respective side of the eye mask.

3. The travel pillow of claim 2 wherein the neck pillow includes an upper section which is thin and pliable, and a lower section which is filled with a stuffing material to provide support to the user's neck.

4. The travel pillow of claim 1 wherein the neck pillow includes an upper section which is thin and pliable, and a lower section which is filled with a stuffing material to provide support to the user's neck.

5. The travel pillow of claim 4 wherein each of the ear pillows includes means for retaining an earbud to thereto.

6. The travel pillow of claim 5 wherein the means for retaining comprises a strip of material which is secured to the respective ear pillow at each end of the strip.

7. The travel pillow of claim 1 wherein each of the ear pillows includes means for retaining an earbud to thereto.

8. The travel pillow of claim 7 wherein the means for retaining comprises a strip of material which is secured to the respective ear pillow at each end of the strip.

9. The travel pillow of claim 1 wherein the neck pillow includes an elongated loop having the means for securing passing therethrough.

10. The travel pillow of claim 1 including a pocket on the neck pillow, wherein the eye mask, the means for securing the eye mask to the user's face, and the pair of ear pillows can be folded into the pocket when the travel pillow is not in use.

11. A travel pillow comprising:
(a) a pliable eye mask contoured for securement over a user's eyes;
(b) means for securing the eye mask over the user's eyes; and
(c) at least one neck pillow adjustably secured to the means for securing, the neck pillow including an upper section which is thin and pliable, and a lower section which is filled with a stuffing material to provide support to the user's neck.

12. The travel pillow of claim 11 wherein the means for securing comprises a flexible band having opposed ends, each end of the band being connected to a respective side of the eye mask.

13. The travel pillow of claim 12 including a pair of ear pillows, each ear pillow being adjustably secured to the means for securing and positioned on a respective side of the eye mask such that the ear pillows conceal the user's ears when the eye mask is worn by the user.

14. The travel pillow of claim 11 including a pair of ear pillows, each ear pillow being adjustably secured to the means for securing and positioned on a respective side of the eye mask such that the ear pillows conceal the user's ears when the eye mask is worn by the user.

15. The travel pillow of claim 14 wherein each of the ear pillows includes means for retaining an earbud to thereto.

16. The travel pillow of claim 15 wherein the means for retaining comprises a strip of material which is secured to the respective ear pillow at each end of the strip.

17. The travel pillow of claim 11 wherein each of the ear pillows includes means for retaining an earbud to thereto.

18. The travel pillow of claim 17 wherein the means for retaining comprises a strip of material which is secured to the respective ear pillow at each end of the strip.

19. The travel pillow of claim 11 wherein the neck pillow includes an elongated loop having the means for securing passing therethrough.

20. The travel pillow of claim 11 including a pocket on the neck pillow, wherein the eye mask, the means for securing the eye mask to the user's face, and the pair of ear pillows can be folded into the pocket when the travel pillow is not in use.
